SEQUENCE LISTING (6 sequences)

SEQ ID NO: 1 - DNA, 5061 bp, Homo sapiens
gacagacatggcgcttcagctgtcttcagaataatgtcacccggcctcctctcctgtcttctgcagtcttaaaagacctagtccctaactgaggtctggcttttcctcagccctggatgaagtggagaagacctatttggagactgcttcctgtcaccataaaatcctgaacatttgtcttgaacatgaacactccaaaagaagaattccaggactggcccatagtcagaatagcagctcatttaccagacctcattgtctatggacatttctccccagagcgaccctttatggattattttgacggagtcctgatgtttgttgatatttcaggttttactgcaatgactgagaagttcagcagtgccatgtacatggacagaggggctgagcagttggtggagatcctcaactaccacataagtgcaatagtggagaaagtgttgatttttggaggagacatcctgaaatttgcaggtgatgcactgctagccctgtggagggtggagcgaaagcagctgaaaaacattatcacagtggtaattaaatgtagcctggagatccatggattgtttgagacccaggagtgggaagaaggcctagacatccgagtcaagataggactggctgctggccacatcagcatgttggtctttggagatgaaacacacagccactttctggtgattggtcaggcagtggacgatgtgcgccttgcccagaacatggctcagatgaatgatgttattctgtcaccaaactgctggcagctctgtgaccggagcatgattgaaattgagagtgttccagatcagagagcagttaaggttaacttcttaaaaccaccccccaattttaattttgatgaatttttcacaaagtgtacgaccttcatgcattattatccttctggtgagcacaaaaacctcctgaggcttgcatgcacgctgaagcctgatcctgaactggagatgtccctacaaaagtatgtgatggaaagcattttgaagcagattgataacaaacagcttcagggctatttatctgagcttcgcccagtgacgattgtgtttgtgaacctgatgtttgaagaccaagacaaagcagaagagataggcccagccatccaggatgcctatatgcacatcacttctgtcctgaagatcttccaaggccaaatcaataaagtcttcatgtttgacaagggctgctctttcctctgtgtctttggcttccctggggaaaaggtacctgacgagctcactcatgctctggaatgtgctatggatatatttgacttctgctctcaagtccacaaaatccaaactgtatccatcggtgttgccagtgggattgtcttctgtgggatcgttggacacactgtgagacacgagtacacagtcattggtcaaaaagtcaacttagctgccaggatgatgatgtactacccaggaattgtgacctgcgactctgtcacctacaatgggagcaacctaccagcgtacttttttaaagagcttccaaagaaagttatgaaaggtgttgcagattctggaccattgtatcagtattggggccgtactgagaaagtcatgtttggtatggcgtgcctcatctgcaacagaaaggaggattaccctttgctgggacgtaataaagagatcaactacttcatgtatactatgaagaaatttttgatatctaacagcagccaagtcttaatgtatgagggattaccaggatatggaaaaagccagatacttatgaaaattgagtacctggcccaaggtaagaatcacaggattattgccatttcattgaataagatcagcttccatcaaactttctataccatccagatgttcatggccaatgtcctaggcctagacacttgtaaacattataaagaacgacagaccaaccttcgaaataaagtcatgacactgttggatgaaaagttctactgtcttcttaatgacattttccatgttcagttccctatttctcgggagatttccaggatgagcaccttgaaaaagcaaaaacaattggaaatattgtttatgaagatcttgaagctgatagtgaaagaggaaaggattatttttatcattgatgaggcccagtttgtggattcgacctcctggagatttatggagaagcttatccggactcttcctatcttcatcattatgtccctgtgtcccttcgttaacattccctgtgcagctgccagggccgtaataaagaacaggaacaccacctacattgtcattggtgcagtacagcctaacgacatctccaacaagatctgtcttgacctcaatgtgagctgcatctccaaagaactggactcgtacctgggggagggaagctgtgggattccattttactgtgaagaattgcttaaaaacctggaacatcatgaggtactcgttttccaacaaacggagtctgaggaaaagacaaataggacctggaataacctgttcaagtattccattaagctaacagagaagttaaacatggttactctccatagtgataaggaaagtgaagaagtctgtcacctcacaagtggtgtcagactgaaaaacctgtcacctccaacgtcattaaaagaaatctctctgatccagctggatagcatgagactttcccaccaaatgctggtgagatgtgctgccatcattggcctgaccttcaccactgagttgttgtttgagattctcccctgttggaatatgaagatgatgatcaagaccctggcaaccctagtggaatctaacattttttattgtttccggaatggcaaggagcttcaaaaggccctgaaacagaatgatccctcatttgaggtgcactatcgttccttgtctctgaagcccagtgaagggatggatcacggtgaagaggaacagcttcgtgaactggagaatgaggtgatcgagtgccacaggattcgattctgtaaccctatgatgcagaaaacagcctacgagctgtggctcaaggaccagagaaaagccatgcacttgaaatgtgcccgctttttagaagaagatgcccacagatgtgaccactgccgaggcagggacttcattccctatcatcacttcacagtgaatattcggctcaacgctttagacatggatgccattaaaaagatggctatgtctcatggatttaaaactgaagaaaagcttatcttgtccaactcagagattcctgagacatctgcattttttcctgaaaatcgcagtcctgaagaaataagagaaaagatcttgaatttctttgaccacgttttaacaaaaatgaagacatctgacgaagacattatccctctggaatcttgccagtgtgaagaaatcctagagattgtcatcttgcctctggcccaccattttctggctttgggagaaaatgacaaagccttatattacttcttagaaattgcatctgcttatctcatcttttgtgataactacatggcatacatgtatttgaatgaaggacagaagttgctaaaaactctcaagaaggacaaatcttggagccagacatttgagtctgccaccttttacagcctcaaaggtgaggtctgtttcaatatgggccagatagtgcttgccaagaaaatgctgaggaaggcactgaagctcctcaaccgaatctttccttacaacttaatctccttgtttctccatatccatgtcgagaaaaacagacactttcattatgtgaatcggcaggcccaagagagcccacctccagggaagaagaggctggcacaactttaccggcaaactgtctgcctttccttgctgtggcgcatctatagctacagttatctttttcactgcaagtattatgcccacctggcagttatgatgcaaatgaatactgcactggaaactcaaaattgtttccagatcattaaggcttacctagactattcgctataccaccacctggctggctacaaaggtgtgtggttcaaatatgaagtcatggccatggagcacatcttcaacctccccctgaaaggcgagggcattgaaatcgtggcatacgtggctgagacactggtcttcaacaagctcataatgggacacctggatttggccattgagttaggctcccgagcccttcagatgtgggcactgctccagaatcccaaccgacattatcagtccctctgcagacttagcagatgtctccttctgaacagtagatacccgcaattgatccaggtgccggggcggctgtgggagctttctgtaacacaggaacacatcttcagcaaggcatttttctattttgtctgcttggacatcctgctttattctggttttgtttatagaacatttgaagaatgtttggaattcatacaccaatacgaaaacaacagaatcctcaagttccacagtggactcctcctgggactttattcctctgtagctatctggtatgccagacttcaggaatgggacaacttttacaaattttccaatagagctaaaaatcttttgccaagaagaaccatgacacttacttactatgacggaatatctaggtacatggaggggcaagttcttcaccttcaaaaacaaatcaaagaacagtcagagaatgcccaagccagtggggaggagctactcaagaacttggagaatctggtggctcaaaataccactggccctgtcttttgcccaaggctctaccacctgatggcttacgtctgtatattaatgggagatgggcagaaatgtggcctcttcctgaacacagccttgcggctctctgaaacacaggggaatatactggagaaatgctggctgaacatgaacaaagaatcatggtactcaacctctgagttaaaagaagaccaatggcttcagacgatcttgagtctcccatcatgggaaaaaattgtagcaggcagggtaaacattcaggatcttcaaaaaaacaaattcctgatgagagctaataccgtggacaatcatttctaacatgtcaaagaaaaaagattttaataagcactaaaaaaaaaaa

SEQ ID NO: 2 - DNA, 5009 bp, Homo sapiens
ggcgcttcagctgtcttcagaataatgtcacccggcctcctctcctgtcttctgcagtcttaaaagacctagtccctaactgaggtctggcttttcctcagccctggatgaagtggagaagacctatttggagactgcttcctgtcaccataaaatcctgaacatttgtcttgaacatgaacactccaaaagaagaattccaggactggcccatagtcagaatagcagctcatttaccagacctcattgtctatggacatttctccccagagcgaccctttatggattattttgacggagtcctgatgtttgttgatatttcaggttttactgcaatgactgagaagttcagcagtgccatgtacatggacagaggggctgagcagttggtggagatcctcaactaccacataagtgcaatagtggagaaagtgttgatttttggaggagacatcctgaaatttgcaggtgatgcactgctagccctgtggagggtggagcgaaagcagctgaaaaacattatcacagtggtaattaaatgtagcctggagatccatggattgtttgagacccaggagtgggaagaaggcctagacatccgagtcaagataggactggctgctggccacatcagcatgttggtctttggagatgaaacacacagccactttctggtgattggtcaggcagtggacgatgtgcgccttgcccagaacatggctcagatgaatgatgttattctgtcaccaaactgctggcagctctgtgaccggagcatgattgaaattgagagtgttccagatcagagagcagttaaggttaacttcttaaaaccaccccccaattttaattttgatgaatttttcacaaagtgtacgaccttcatgcattattatccttctggtgagcacaaaaacctcctgaggcttgcatgcacgctgaagcctgatcctgaactggagatgtccctacaaaagtatgtgatggaaagcattttgaagcagattgataacaaacagcttcagggctatttatctgagcttcgcccagtgacgattgtgtttgtgaacctgatgtttgaagaccaagacaaagcagaagagataggcccagccatccaggatgcctatatgcacatcacttctgtcctgaagatcttccaaggccaaatcaataaagtcttcatgtttgacaagggctgctctttcctctgtgtctttggcttccctggggaaaaggtacctgacgagctcactcatgctctggaatgtgctatggatatatttgacttctgctctcaagtccacaaaatccaaactgtatccatcggtgttgccagtgggattgtcttctgtgggatcgttggacacactgtgagacacgagtacacagtcattggtcaaaaagtcaacttagctgccaggatgatgatgtactacccaggaattgtgacctgcgactctgtcacctacaatgggagcaacctaccagcgtacttttttaaagagcttccaaagaaagttatgaaaggtgttgcagattctggaccattgtatcagtattggggccgtactgagaaagtcatgtttggtatggcgtgcctcatctgcaacagaaaggaggattaccctttgctgggacgtaataaagagatcaactacttcatgtatactatgaagaaatttttgatatctaacagcagccaagtcttaatgtatgagggattaccaggatatggaaaaagccagatacttatgaaaattgagtacctggcccaaggtaagaatcacaggattattgccatttcattgaataagatcagcttccatcaaactttctataccatccagatgttcatggccaatgtcctaggcctagacacttgtaaacattataaagaacgacagaccaaccttcgaaataaagtcatgacactgttggatgaaaagttctactgtcttcttaatgacattttccatgttcagttccctatttctcgggagatttccaggatgagcaccttgaaaaagcaaaaacaattggaaatattgtttatgaagatcttgaagctgatagtgaaagaggaaaggattatttttatcattgatgaggcccagtttgtggattcgacctcctggagatttatggagaagcttatccggactcttcctatcttcatcattatgtccctgtgtcccttcgttaacattccctgtgcagctgccagggccgtaataaagaacaggaacaccacctacattgtcattggtgcagtacagcctaacgacatctccaacaagatctgtcttgacctcaatgtgagctgcatctccaaagaactggactcgtacctgggggagggaagctgtgggattccattttactgtgaagaattgcttaaaaacctggaacatcatgaggtactcgttttccaacaaacggagtctgaggaaaagacaaataggacctggaataacctgttcaagtattccattaagctaacagagaagttaaacatggttactctccatagtgataaggaaagtgaagaagtctgtcacctcacaagtggtgtcagactgaaaaacctgtcacctccaacgtcattaaaagaaatctctctgatccagctggatagcatgagactttcccaccaaatgctggtgagatgtgctgccatcattggcctgaccttcaccactgagttgttgtttgagattctcccctgttggaatatgaagatgatgatcaagaccctggcaaccctagtggaatctaacattttttattgtttccggaatggcaaggagcttcaaaaggccctgaaacagaatgatccctcatttgaggtgcactatcgttccttgtctctgaagcccagtgaagggatggatcacggtgaagaggaacagcttcgtgaactggagaatgaggtgatcgagtgccacaggattcgattctgtaaccctatgatgcagaaaacagcctacgagctgtggctcaaggaccagagaaaagccatgcacttgaaatgtgcccgctttttagaagaagatgcccacagatgtgaccactgccgaggcagggacttcattccctatcatcacttcacagtgaatattcggctcaacgctttagacatggatgccattaaaaagatggctatgtctcatggatttaaaactgaagaaaagcttatcttgtccaactcagagattcctgagacatctgcattttttcctgaaaatcgcagtcctgaagaaataagagaaaagatcttgaatttctttgaccacgttttaacaaaaatgaagacatctgacgaagacattatccctctggaatcttgccagtgtgaagaaatcctagagattgtcatcttgcctctggcccaccattttctggctttgggagaaaatgacaaagccttatattacttcttagaaattgcatctgcttatctcatcttttgtgataactacatggcatacatgtatttgaatgaaggacagaagttgctaaaaactctcaagaaggacaaatcttggagccagacatttgagtctgccaccttttacagcctcaaaggtgaggtctgtttcaatatgggccagatagtgcttgccaagaaaatgctgaggaaggcactgaagctcctcaaccgaatctttccttacaacttaatctccttgtttctccatatccatgtcgagaaaaacagacactttcattatgtgaatcggcaggcccaagagagcccacctccagggaagaagaggctggcacaactttaccggcaaactgtctgcctttccttgctgtggcgcatctatagctacagttatctttttcactgcaagtattatgcccacctggcagttatgatgcaaatgaatactgcactggaaactcaaaattgtttccagatcattaaggcttacctagactattcgctataccaccacctggctggctacaaaggtgtgtggttcaaatatgaagtcatggccatggagcacatcttcaacctccccctgaaaggcgagggcattgaaatcgtggcatacgtggctgagacactggtcttcaacaagctcataatgggacacctggatttggccattgagttaggctcccgagcccttcagatgtgggcactgctccagaatcccaaccgacattatcagtccctctgcagacttagcagatgtctccttctgaacagtagatacccgcaattgatccaggtgccggggcggctgtgggagctttctgtaacacaggaacacatcttcagcaaggcatttttctattttgtctgcttggacatcctgctttattctggttttgtttatagaacatttgaagaatgtttggaattcatacaccaatacgaaaacaacagaatcctcaagttccacagtggactcctcctgggactttattcctctgtagctatctggtatgccagacttcaggaatgggacaacttttacaaattttccaatagagctaaaaatcttttgccaagaagaaccatgacacttacttactatgacggaatatctaggtacatggaggggcaagttcttcaccttcaaaaacaaatcaaagaacagtcagagaatgcccaagccagtggggaggagctactcaagaacttggagaatctggtggctcaaaataccactggccctgtcttttgcccaaggctctaccacctgatggcttacgtctgtatattaatgggagatgggcagaaatgtggcctcttcctgaacacagccttgcggctctctgaaacacaggggaatatactggagaaatgctggctgaacatgaacaaagaatcatggtactcaacctctgagttaaaagaagaccaatggcttcagacgatcttgagtctcccatcatgggaaaaaattgtagcaggcagggtaaacattcaggatcttcaaaaaaacaaattcctgatgagagctaataccgtggacaatcatttctaa

SEQ ID NO: 3 - protein, 1610 aa, Homo sapiens [sequence omitted]

SEQ ID NO: 4 - protein, 411 aa, Homo sapiens
LMFVDISGFTAMTEKFSSAMYMDRGAEQLVEILNYHISAIVEKVLIFGGDILKFAGDALLALWRVERKQLKNIITVVIKCSLEIHGLFETQEWEEGLDIRVKIGLAAGHISMLVFGDETHSHFLVIGQAVDDVRLAQNMAQMNDVILSPNCWQLCDRSMIEIESVPDQRAVKVNFLKPPPNFNFDEFFTKCTTFMHYYPSGEHKNLLRLACTLKPDPELEMSLQKYVMESILKQIDNKQLQGYLSELRPVTIVFVNLMFEDQDKAEEIGPAIQDAYMHITSVLKIFQGQINKVFMFDKGCSFLCVFGFPGEKVPDELTHALECAMDIFDFCSQVHKIQTVSIGVASGIVFCGIVGHTVRHEYTVIGQKVNLAARMMMYYPGIVTCDSVTYNGSNLPAYFFKELPKKVMKGV

SEQ ID NO: 5 - protein, 16 aa, Homo sapiens
CTLKPDPELEMSLQKY

SEQ ID NO: 6 - DNA, 1224 bp, Homo sapiens
atgtttgttgatatttcaggttttactgcaatgactgagaagttcagcagtgccatgtacatggacagaggggctgagcagttggtggagatcctcaactaccacataagtgcaatagtggagaaagtgttgatttttggaggagacatcctgaaatttgcaggtgatgcactgctagccctgtggagggtggagcgaaagcagctgaaaaacattatcacagtggtaattaaatgtagcctggagatccatggattgtttgagacccaggagtgggaagaaggcctagacatccgagtcaagataggactggctgctggccacatcagcatgttggtctttggagatgaaacacacagccactttctggtgattggtcaggcagtggacgatgtgcgccttgcccagaacatggctcagatgaatgatgttattctgtcaccaaactgctggcagctctgtgaccggagcatgattgaaattgagagtgttccagatcagagagcagttaaggttaacttcttaaaaccaccccccaattttaattttgatgaatttttcacaaagtgtacgaccttcatgcattattatccttctggtgagcacaaaaacctcctgaggcttgcatgcacgctgaagcctgatcctgaactggagatgtccctacaaaagtatgtgatggaaagcattttgaagcagattgataacaaacagcttcagggctatttatctgagcttcgcccagtgacgattgtgtttgtgaacctgatgtttgaagaccaagacaaagcagaagagataggcccagccatccaggatgcctatatgcacatcacttctgtcctgaagatcttccaaggccaaatcaataaagtcttcatgtttgacaagggctgctctttcctctgtgtctttggcttccctggggaaaaggtacctgacgagctcactcatgctctggaatgtgctatggatatatttgacttctgctctcaagtccacaaaatccaaactgtatccatcggtgttgccagtgggattgtcttctgtgggatcgttggacacactgtgagacacgagtacacagtcattggtcaaaaagtcaacttagctgccaggatgatgatgtactacccaggaattgtgacctgcgactctgtcacctacaatgggagcaacctaccagcgtacttttttaaagagcttccaaagaaagttatgaaa

(19) United States
(12) Patent Application Publication
(10) Pub. No.: US 2002/0064849 A1
(43) Pub. Date: May 30, 2002

(54) Human soluble testicular adenylyl cyclase

(76) Inventors: John C. Herr, Charlottesville, VA (US); Pablo Visconti, Charlottesville, VA (US); Arabinda Mandal, Charlottesville, VA (US); Vrinda Khole, Worli (IN)

Correspondence Address: John Breen, University of Virginia Patent Foundation, Suite 1-110, 1224 West Main Street, Charlottesville, VA, 22903, US

(21) Appl. No.: 09/947,124

(22) Filed: Sep. 5, 2001

Related U.S. Application Data
(60) Provisional application No. 60/230,207, filed on Sep. 5, 2000.

(51) Int. Cl. C12Q 1/42; C07H 21/04; A61K 38/47; C12N 9/16; C07K 16/40; C12P 21/02; C12N 5/06

(52) U.S. Cl. 435/190; 435/69.1; 435/325; 530/388.26; 435/21; 536/23.2; 424/94.61

(57) ABSTRACT

The present invention relates to a newly identified human soluble adenylyl cyclase and nucleic acid sequences encoding the adenylyl cyclase. The invention further relates to methods of using the adenylyl cyclase polypeptides and polynucleotides as a targets for identifying agonists and antagonists that are selective for human soluble adenylyl cyclase. Inhibitors of human soluble adenylyl cyclase can be used as contraceptive agents.

This application claims priority under 35 U.S.C. §119(e) to provisional patent application No. 60/230,207, filed Sep. 5, 2000, the disclosure of which is incorporated herein.

### US GOVERNMENT RIGHTS

This invention was made with United States Government support under Grant No. HD U54 29099, P30 28934, T32 DK 07641, T32 HD 07382 U54 HD 28934, HG 00333 and D43 TW/HD 00654 awarded by the National Institutes of Health. The United States Government has certain rights in the invention.

### FIELD OF THE INVENTION

The present invention is directed to a novel human soluble adenylyl cyclase that is expressed in the testis. The invention also encompasses nucleic acid sequences that encode the novel soluble human adenylyl cyclase.

### BACKGROUND OF THE INVENTION

Mammalian sperm are not able to fertilize eggs immediately after ejaculation. Fertilization capacity is acquired by spermatozoa only after residence in the distinct microenvironments of the uterus or oviduct (depending on the species) for a finite period of time. The necessary series of changes, termed capacitation, was first described independently by Chang and Austin in the early to mid 1950s. Capacitation involves molecular changes in both the sperm head and tail which allow defined physiological endpoints to occur such as motility hyperactivation, a whiplash-like sperm tail motion, and regulated acrosomal exocytosis. Hyperactivation is observed when sperm reach the oocyte and increase their flagellar bend amplitude and beat asymmetry which are thought to enhance the ability of sperm to penetrate the egg vestments by increasing forward progression and lateral flagellar thrust.

Using the mouse as an experimental model, capacitation has been demonstrated to be regulated by a cAMP-dependent pathway involving protein kinase A (PKA) (Visconti, Galantino-Homer et al. 1999, J Biol Chem 274(5): 3235-42.; Visconti, Ning et al. 1999 Dev Biol 214(2): 429-43; Visconti, Stewart-Savage et al. 1999 Biol Reprod 61(1):76-84). The presence of this regulatory pathway has subsequently been demonstrated in sperm from other species such as bovine, human, boar and hamster. Besides its function in capacitation, the role of cAMP in sperm motility has been well established (Eddy and O'Brien (1994). The Spermatozoon. The Physiology of Reproduction. E. Knobil and J. D. Neill. New York, Raven Press. 1:29-77). Cytosolic levels of cAMP increase during capacitation, and pharmacological stimulants which elevate intracellular cAMP such as the phosphodiesterase inhibitors, caffeine and pentoxifylline enhance sperm hyperactivated motility, enhance penetration of cervical mucus, increase tight binding to homologous zona pellucida, and increase fertilization rates.

Despite the advances in the understanding of cAMP metabolism in sperm, little is known about the identity and properties of the enzyme responsible for cAMP synthesis in mammalian sperm. In mammals, nine distinct adenylyl cyclase genes (“somatic adenylyl cyclases”) have been isolated and sequenced and their pattern of expression and regulatory properties have thus far been identified. The synthesis of cAMP by these somatic adenylyl cyclases is regulated by G proteins and other signaling molecules in response to stimuli such as hormones and neurotransmiters. Sperm adenylyl cyclases have different properties when compared to their somatic counterparts and these differing properties suggest that a unique isoform of adenylyl cyclase exists in mammalian sperm. In particular, some of the distinguishing properties between sperm and somatic adenylyl cyclases include:

1) The activity ratio when measured in the presence of Mn2+or Mg2+is from 1 to 2 in somatic cyclases and from 10 to 20 in mammalian sperm cyclases.

2) Somatic adenylyl cyclases are regulated by the stimulatory G protein and can be stimulated by A1IF4-, cholera toxin and GTP analogues, sperm cyclase activity is independent of these factors.

3) Forskolin is a well known activator of somatic adenylyl cyclases and is not active (or only slightly active) for mammalian sperm adenylyl cyclases.

4) Sperm adenylyl cyclase activity is the only one that can be stimulated by bicarbonate anion. No somatic cyclase respond to this anion in vivo or in vitro.

Until the present invention, all attempts to clone the human sperm adenylyl cyclase have failed. For example, the use of degenerated oligonucleotides (based on sequences with high homology between different somatic cyclases) in a RT-PCR failed isolate a sperm cyclase. One reason for this failure could be that unique catalytic domains are present in the sperm enzyme. Recently, Buck et al. (1999) Proc Natl Acad Sci USA 96(1):79-84 have purified and cloned from rat testis a soluble isoform of adenylyl cyclase. These authors have demonstrated that this enzyme is mainly expressed in testicular germ cells and they have recently shown that this enzyme is also present in mature sperm.

The same group has demonstrated that recombinant rat SAC can be stimulated by bicarbonate and that antibodies directed against the catalytic domain of SAC recognized proteins in testis, sperm, kidney and choroid plexus (Chen, Cann et al. 2000). However, these results have not yet been validated by microsequence of the proteins recognized by the anti SAC antibody. This is an important validation since: 1) the amount of protein from kidney and choroid plexus tissue used by Chen and coworkers was 10 times the amount of protein from sperm in the same experiment. 2) In testis and sperm, the antibody recognized a protein of high molecular weight as well as one with a lower mass. In choroid plexus and kidney the only protein recognized has a low molecular weight. 3) the cyclase assay that follows anti SAC immunoprecipitation was performed exclusively in testis extracts and not in sperm, kidney or choroid plexus. In addition there has been no report of the isolation or cloning of the human homologue of the soluble adenylyl cyclase.

The present invention is directed to the isolation and characterization of the human soluble sperm adenylyl cyclase and nucleic acids sequences encoding the same. The present invention also provides methods of screening for compounds that modulate the expression or activity of the human SAC nucleic acids (DNA or RNA) or polypeptide, respectively. In one embodiment a method is provided for identifying compounds that selectively inhibit the expression or activity of human SAC. Such inhibitory compounds can be used in pharmaceutical formulations to inhibit the ability of sperm cells to fertilize an ovum and thus provide ideal candidates as non-hormonal contraceptive agents.

Definitions

In describing and claiming the invention, the following terminology will be used in accordance with the definitions set forth below.

As used herein, “nucleic acid,” “DNA,” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

The term “peptide” encompasses a sequence of 3 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR—linkages (bonds) have been replaced by a non-peptidyl linkage such as a —CH₂₋carbamate linkage (—CH₂OC(O)NR—), a phosphonate linkage, a —CH₂₋sulfonamide (—CH_{2—}S(O)₂NR—) linkage, a urea (—NHC(O)NH—) linkage, a —CH₂-secondary amine linkage, or with an alkylated peptidyl linkage (—C(O)NR—) wherein R is C₁₋C₄ alkyl;

2. peptides wherein the N-terminus is derivatized to a —NRR₁ group, to a —NRC(O)R group, to a —NRC(O)OR group, to a —NRS(O)₂R group, to a —NHC(O)NHR group where R and R₁ are hydrogen or C₁₋C₄ alkyl with the proviso that R and R₁ are not both hydrogen;

3. peptides wherein the C terminus is derivatized to —C(O)R₂ where R₂ is selected from the group consisting of C ₁-C₄ alkoxy, and —NR₃R₄ where R₃ and R₄ are independently selected from the group consisting of hydrogen and C₁₋C₄ alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Vat or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for trytophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” are defined herein as exchanges within one of the following five groups:

I. Small aliphatic, nonpolar or slightly polar residues:

Ala, Ser, Thr, Pro, Gly;

II. Polar, negatively charged residues and their amides:

Asp, Asn, Glu, Gln;

III. Polar, positively charged residues:

His, Arg, Lys;

IV. Large, aliphatic, nonpolar residues:

Met Leu, Ile, Val, Cys

V. Large, aromatic residues:

Phe, Tyr, Trp

As used herein, the term “isolate” and like terms relate to the purification of a molecule or compound in a form that is substantially free of contaminants normally associated with the molecule or compound in a native or natural environment.

As used herein, the term “human soluble adenylyl cyclase” or “human SAC” and like terms refers to polypeptides comprising SEQ ID NO: 3 and biologically active derivatives or fragments thereof.

As used herein, the term “biologically active derivative or fragment” or “bioactive derivative or fragment” of a human soluble adenylyl cyclase encompasses natural or synthetic portions of SEQ ID NO: 3 as well as modified versions of the SEQ ID NO: 3 polypeptide that contain multiple conservative amino acid substitutions, wherein the derivative or fragment polypeptide exhibits adenylyl cyclase activity (i.e. are capable of catalyzing the formation of cyclic adenosine monophosphate (cAMP) from adenosine triphosphate (ATP)).

“Operably linked” refers to a juxtaposition wherein the components are configured so as to perform their usual function. Thus, control sequences or promoters operably linked to a coding sequence are capable of effecting the expression of the coding sequence.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

### SUMMARY OF THE INVENTION

The present invention is directed to the isolation and characterization of a novel human soluble adenylyl cyclase and nucleic acid sequences encoding the same. More particularly, the present invention is directed to an isolated polypeptide comprising an amino acid sequence selected from the group consisting of SEQ ID NO: 3 or SEQ ID NO:4 or a derivative thereof, wherein the adenylyl cyclase is expressed in male germ line cells and the cyclase activity is stimulated by bicarbonate anion. The adenylyl cyclase of the present invention provides a useful target for identifying compounds that specifically inhibit cyclase activity in male germ line cells, thus identifying potential contraceptive agents.

BRIEF DESCRIPTION OF THE DRAWINGS

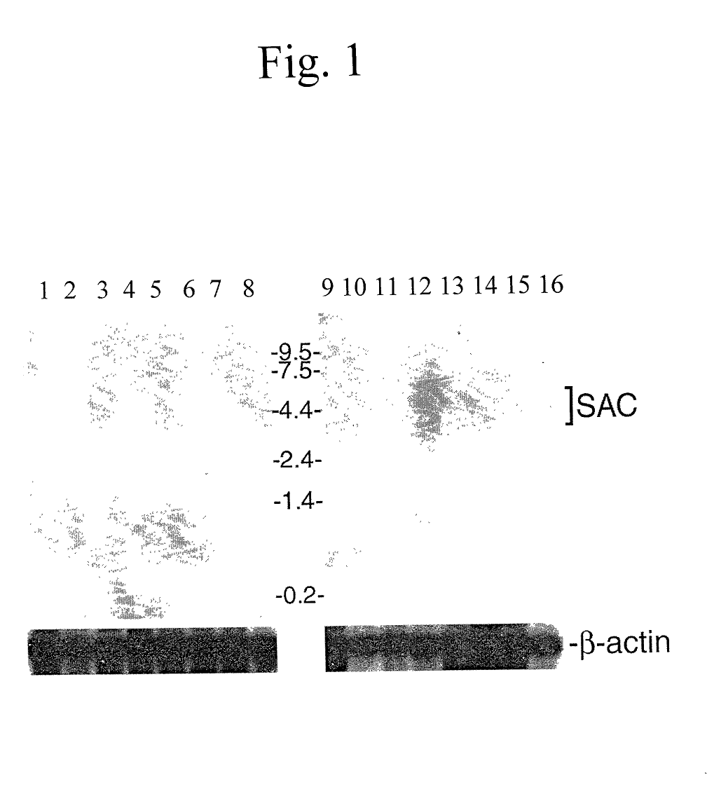

FIG. 1 Expression pattern of the human homologue of SAC in various tissues. A Northern blot was performed using a commercially available human tissue blot (Clontech, Lane1=heart; Lane 2=brain; Lane 3=placenta; Lane 4=lung; Lane 5=liver; Lane 6=skeletal muscle; Lane 7=kidney; Lane 8=pancreas; Lane 9=spleen; Lane 10=thymus; Lane 11=prostate; Lane 12=testis; Lane 13=ovary; Lane 14=small intestine; Lane 15=colon; Lane 16=PBLs). cDNA containing the catalytic domain (SEQ ID NO: 4) was random primed-radiolabeled and hybridized to Clontech tissue northern blot. The hybridized membrane was exposed for 4 days, exposure for longer time did not show other transcripts. The lower panel represents the same northern blot probed with an actin specific sequence.

### DETAILED DESCRIPTION OF THE INVENTION

Despite the availability of a large range of contraceptive methods, in the U.S. alone, over 50% of pregnancies are unintended. Thus, there is a critical need for contraception that better fits the diverse needs of women and men and take into consideration different ethnic, cultural and religious values. Development of effective, safe and acceptable contraceptive drugs is a major component of the women's health agenda. In this respect, the soluble testicular cyclase offers new opportunities as a novel target for contraception.

The present invention is directed to the isolation a novel human soluble adenylyl cyclase (human SAC) and its use to identify inhibitors of human SAC that can be used as contraceptive agents. Human SAC's suitability as a target for identifying new contraceptive agents derives from several general properties of testicular soluble adenylyl cyclases. First, from published reports (Buck, Sinclair et al. 1999; Sinclair, Wang et al. 2000) and data presented herein, this enzyme appears to be mainly expressed in germ cells and in placenta, as seen by northern blotting. Second its sequence is unique and has very little homology to other somatic adenylyl cyclases, which supports the likelihood of finding specific inhibitors for its activity. Third, the importance of cAMP for sperm function suggests that the inhibition of its synthesis with a specific drug will inhibit fertilization. Finally, the fact that adenylyl cyclase activity is essential for sperm motility and capacitation suggests that an inhibitor of sperm adenylyl cyclase could be used both in male and female in order to interrupt fertilization.

One aspect of the present invention is directed to human soluble adenylyl cyclase (human SAC) protein itself and the nucleic acid sequences encoding the enzyme. More particularly, the present invention is directed to an isolated polypeptide comprising the amino acid sequence of SEQ ID NO: 3 or SEQ ID NO: 4, or an amino acid sequence that differs from SEQ ID NO: 3 by one or more conservative amino acid substitutions. In one embodiment, the isolated polypeptide comprises an amino acid sequence that differs from SEQ ID NO: 3 by 20 or less conservative amino acid substitutions, and more preferably by 10 or less conservative amino acid substitutions, and retains adenylyl cyclase activity. Alternatively, the polypeptide may comprise an amino acid sequence that differs from SEQ ID NO: 3 by 1 to 5 alterations, wherein the alterations are independently selected from a single amino acid deletion, insertion or substitution.

The present invention also encompasses nucleic acid sequences that encode the human soluble adenylyl cyclase. In particular the present invention is directed to nucleic acid sequences comprising the sequence of SEQ ID NO: 1 or fragments thereof. In one embodiment, an isolated nucleic acid is provided that comprises at least 50 (contiguous) nucleotides, 100 nucleotides, 200 nucleotides, or 500 nucleotides of SEQ ID NO: 2. In one embodiment the nucleic acid sequence consists of the sequence of SEQ ID NO: 2.

The present invention also includes nucleic acids that hybridize (under conditions defined herein) to all or a portion of the nucleotide sequence represented by SEQ ID NO:1 or its complement. The hybridizing portion of the hybridizing nucleic acids is typically at least 15 (e.g., 20, 25, 30, or 50) nucleotides in length. Hybridizing nucleic acids of the type described herein can be used, for example, as a cloning probe, a primer (e.g., a PCR primer), or a diagnostic probe to detect the expression of the human SAC gene. It is anticipated that the DNA sequence of SEQ ID NO: 2, or fragments thereof can be used to distinguish between the expression of somatic and soluble adenylyl cyclase genes or as probes to detect homologous genes from other vertebrate species.

Nucleic acid duplex or hybrid stability is expressed as the melting temperature or Tm, which is the temperature at which a nucleic acid duplex dissociates into its component single stranded DNAs. This melting temperature is used to define the required stringency conditions. Typically a 1% mismatch results in a 1° C. decrease in the Tm, and the temperature of the final wash in the hybridization reaction is reduced accordingly (for example, if two sequences having >95% identity, the final wash temperature is decreased from the Tm by 5° C.). In practice, the change in Tm can be between 0.5° C. and 1.5° C. per 1% mismatch.

The present invention is directed to the nucleic acid sequence of SEQ ID NO: 2 and nucleic acid sequences that hybridize to that sequence (or fragments thereof) under stringent or highly stringent conditions. In accordance with the present invention highly stringent conditions are defined as conducting the hybridization and wash conditions at no lower than −5° C. Tm. Stringent conditions are defined as involve hybridizing at 68° C. in 5×SSC/5×Denhardt's solution/1.0% SDS, and washing in 0.2×SSC/0.1% SDS at 68° C. Moderately stringent conditions include hybridizing at 68° C. in 5×SSC/5×Denhardt's solution/1.0% SDS and washing in 3×SSC/0.1% SDS at 42° C. Additional guidance regarding such conditions is readily available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

The present invention is also directed to amino acid sequences that are variants of the amino acid sequence of SEQ ID NO: 3, wherein the variant is encoded by a nucleic acid sequence that hybridizes to the nucleic acid sequence of SEQ ID NO: 2 under stringent conditions or highly stringent conditions. Such polypeptides are anticipated to include allelic variants of the polypeptide of SEQ ID NO: 3.

In another embodiment of the present invention, nucleic acid sequences encoding the human soluble adenylyl cyclase can be inserted into expression vectors and used to transfect cells to produce transgenic cells. In accordance with one embodiment, nucleic acid sequences encoding human soluble adenylyl cyclase are inserted into a eukaryotic expression vector in a manner that operably links the gene sequences to the appropriate regulatory sequences, and human soluble adenylyl cyclase is expressed in a eukaryotic or prokaryotic cells host cell. Suitable eukaryotic host cells and vectors are known to those skilled in the art. In one embodiment the nucleic acid sequence to be operably linked to the expression vector regulatory sequences is selected from the group consisting of SEQ ID NO: 2 and SEQ ID NO: 6. One aspect of the present invention is directed to transgenic cell lines that contain recombinant genes that express human soluble adenylyl cyclase and fragments of the human SAC coding sequence. The present invention also includes non-human transgenic organisms wherein one or more of the cells of the transgenic organism comprise a recombinant gene that expresses the human soluble cyclase.

The present invention also encompasses a method for producing human SAC. The method comprises the steps of introducing a nucleic acid sequence comprising sequences encoding the human SAC into a host cell, and culturing the host cell under conditions that allow for expression of the introduced human SAC gene. In one preferred embodiment the nucleic acid sequence comprises the sequence of SEQ ID NO: 2, or a sequence that binds to SEQ ID NO: 2 under stringent conditions, operably linked to a promoter. In one embodiment the promoter is a conditional or inducible promoter, alternatively the promoter may be a tissue specific or temporal restricted promoter (i.e. operably linked genes are only expressed in a specific tissue or at a specific time).

In accordance with one embodiment a composition is provided comprising a peptide having the sequence of SEQ ID NO: 3 or an antigenic fragment thereof. In one embodiment the antigenic fragment consists of the sequence of SEQ ID NO: 4. The compositions can be combined with a pharmaceutically acceptable carrier or adjuvants and administered to a mammalian species to induce an immune response.

Another embodiment of the present invention is directed to the isolated antibodies that are generated against human soluble adenylyl cyclase or fragments thereof. In accordance with the present invention antibodies are provided that bind to a polypeptide selected from the group consisting of SEQ ID NO: 3 and SEQ ID NO: 4. These antibodies can be formulated with standard carriers and optionally labeled to prepare therapeutic or diagnostic compositions. Antibodies to human soluble adenylyl cyclase may be generated using methods that are well known in the art. Such antibodies may include, but are not limited to, polyclonal, monoclonal, chimeric (i.e “humanized” antibodies), single chain (recombinant), Fab fragments, and fragments produced by a Fab expression library. These antibodies can be used to confirm the cellular expression of human soluble adenylyl cyclase, or in assays to monitor patients being treated with human soluble adenylyl cyclase antagonists or inhibitors. The antibodies may be used with or without modification, and may be labeled by joining them, either covalently or non-covalently, with a reporter molecule.

In accordance with one embodiment an antibody is provided that specifically binds to the protein of SEQ ID NO: 3. More preferably, the antibody binds to the human soluble adenylyl cyclase catalytic domain of SEQ ID NO: 4. In one preferred embodiment the antibody is a monoclonal antibody. The present invention also provides a method for detecting the presence of human soluble adenylyl cyclase. The method comprises the steps of contacting a sample with a labeled compound that specifically binds to human SAC, removing unbound and non-specific bond material and detecting the presence of the labeled compound. In one embodiment the labeled compound comprises an antibody that is labeled directly or indirectly (i.e. via a labeled secondary antibody).

One aspect of the present invention is directed to therapeutic and diagnostic methods and compositions based on human SAC proteins and nucleic acids. Alterations in cAMP signal transduction pathway have been associated with diseases such asthma, cancer, inflamation, hypertension, atherosclerosis and heart failure. The identification of the present novel andenylate cyclase allows for the identification of compounds that specifically modulate cyclase activity associated with a particular disease state. In one embodiment, the present invention provides methods of screening for agents, small molecules, or proteins that interact with human SAC. In particular, the present invention is directed to methods of identifying inhibitors of human SAC activity. The invention encompasses both in vivo and in vitro assays to screen small molecules, compounds, recombinant proteins, peptides, nucleic acids, antibodies etc. that modulate the activity of human SAC and are thus useful as contraceptive agents. Preferably, the method of screening for inhibitors of human SAC utilizes high throughput technology.

In one embodiment the human SAC polypeptide, or bioactive fragments thereof, is used to isolate ligands that bind to the human SAC polypeptide under physiological conditions. The method comprises the steps of contacting the human SAC polypeptide with a mixture of compounds under physiological conditions, removing unbound and non-specifically bound material, and isolating the compounds that remain bound to the human SAC polypeptides. Typically, the human SAC polypeptide will be bound to a solid support using standard techniques to allow rapid screening compounds. The solid support can be selected from any surface that has been used to immobilize biological compounds and includes but is not limited to polystyrene, agarose, silica or nitrocellulose. In one embodiment the solid surface comprises functionalized silica or agarose beads. Screening for such compounds can be accomplished using libraries of pharmaceutical agents and standard techniques known to the skilled practitioner.

In accordance with one embodiment, compounds will be isolated based on their ability to suppress or inhibit the expression of the human SAC gene. Preferably the compound will selectively inhibit the human SAC gene, without interfering with the expression of the somatic adenylyl cyclase genes. The method comprises the steps of contacting a cell that expresses the human SAC gene with a potential inhibitor compound, and measuring the expression of the human SAC gene. The expression of the somatic adenylyl cyclases, in the presence and absence of the potential inhibitor will also be investigated.

In one embodiment, compounds will be isolated based on their ability to suppress or inhibit the enzymatic activity of human SAC. The method is based on measuring the amount of cAMP generated in vitro when a solution of ATP and the human adenylyl cyclase are incubated in the presence and absence of a potential inhibitory compound. In particular, the method for detecting compounds that inhibit human soluble adenylyl cyclase activity comprises the steps of contacting human SAC with a potential inhibitory compound, measuring adenylyl cyclase activity in the presence and absence of said compound; and identifying those compounds that decrease the activity of adenylyl cyclase. In one preferred embodiment the human SAC comprises an amino acid sequence selected from the group consisting of SEQ ID NO: 3 and SEQ ID NO: 4, and more particularly, the SAC consists of the amino acid sequence of SEQ ID NO: 3.

Those compounds that exhibit activity as inhibitors of human SAC can be further tested for use as contraceptive agents. For example the inhibitory compounds will be tested to isolate compounds that inhibit human SAC, but fail to substantially inhibit one or more of the somatic adenylyl cyclases. Preferably the cyclase inhibitory compound will decrease human SAC's ability to convert ATP to cAMP without substantially impacting any of the nine somatic adenylyl cyclase's ability to convert ATP to cAMP. cAMP production may be readily measured using methods which are well known in the art, including, for example, methods described by Salomon et al. (Anal. Biochem. 58:541-548, 1976) or Krishna et al. (J. Phamacol. Exp. Ther. 163:379, 1968), or, preferably, using commercially available kits such as the Scintillation Proximity Assay Kit from Amersham Corporation. The Scintillation Proximity Assay Kit measures the production of cAMP by competition of iodinated-cAMP with anti-cAMP antibodies. The amount of adenylyl cyclase activity is then determined based on radioimmunoassay measurements of cAMP formed from ATP.

In one embodiment, the present invention encompasses compositions that can be placed in contact with sperm cells to inhibit the function of the human soluble adenylyl cyclase (i.e. either by inhibiting the expression of human soluble adenylyl cyclase or by interfering with the protein's function). Accordingly, the invention encompasses antibodies, nucleotide constructs and other compounds that inhibit the expression of the human soluble adenylyl cyclase gene (transcription factor inhibitors, antisense and ribozyme molecules, or gene or regulatory sequence replacement constructs) as well as antagonists of cyclase activity.

Compositions comprising an antagonist of human SAC function (i.e. compounds that inhibit expression of the cyclase gene or compounds that interfere with cyclase enzymatic activity) can be used to interfere with the capacitation of vertebrate sperm, and used as contraceptive agents. Furthermore, antibodies against the human SAC protein can be used for the diagnosis of conditions or diseases characterized by expression or overexpression of human SAC, or in assays to monitor patients being treated with human SAC agonists, antagonists or inhibitors.

### EXAMPLE 1

Cloning and Characterization of the Human Homologue of the Rat Soluble Testicular Adenylyl Cyclase

Primers were designed based on the rat SAC sequence and used to PCR amplify human SAC using human testicular cDNA from a Marathon ready human testicular cDNA library (Clontech, Palo Alto,Ca) in a 25 ul assay system for 40 cycles. PCR products were separated on a 1.0% NuSieve agarose gel. The desired DNA fragments from every PCR product were isolated reamplified cloned into the pCR 2.1-TOPO vector. The complementary DNA clones were sequenced in both directions using vector-derived and insert specific primers using a Perkin-Elmer Applied Biosystems DNA sequencer with Big Dye Terminator Chemistry and Taq DNA polymerase. The full length human soluble testicular adenylyl cyclase gene has been sequenced (see SEQ ID NO: 1) and posted in Genbank (accession No #AF299350). The nucleotide and amino acid sequence data were analyzed using GCG programme package and the homology between the rat sequence and the human sequence was determined to be 80% at the nucleotide level.

The full length human SAC cDNA was 5050 bp containing a short 5′ UTR of 185 bp, an ATG encoding the initiator methionine at bp 186, a stop codon TAA at bp 5016, an “alternative” polyadenylation signal at bp 5041-5046, and a polyA tail starting at bp 5051. The SAC cDNA contained an open reading frame of 1610 amino acids encoding a protein estimated at 187 kDa, pI 6.99. The deduced peptide sequence was 77% identical to the rat SAC protein including two highly conserved N-terminal cyclase catalytic domains and an ATP/GTP-binding site motif A (P-loop) at amino acids 516-523. In addition, a C-terminal leucine zipper domain starting at amino acid 1064 and two tetratricopeptide repeat domains (a.a. 1131-1164 and 1511-1544) thought to be involved in protein-protein interactions were identified. The SAC gene locus contained 33 exons and mapped to chromosome 1q24.

Northern blots containing poly (A)+RNA from sixteen human tissues (Lane 1=heart; Lane 2=brain; Lane 3=placenta; Lane 4=lung; Lane 5=liver; Lane 6=skeletal muscle; Lane 7=kidney; Lane 8=pancreas; Lane 9=spleen; Lane 10=thymus; Lane 11=prostate; Lane 12=testis; Lane 13=ovary; Lane 14=small intestine; Lane 15=colon; Lane 16=PBLs) were hybridized with a ³²P labeled probe containing the random primed-radiolabeled 5′ sequence of the human SAC coding region (SEQ ID NO: 2) to determine tissue specificity. As shown in FIG. 1) two transcripts of approximately 4 and 4.4 kb were identified in testis and placenta. An additional transcript of 6 kb hybridized with catalytic SAC in testis, suggesting alternative splicing. The hybridized membrane was exposed for 4 days, exposure for longer time did not show other transcripts. To demonstrate that intact MRNA was present in each tissue, each membrane was stripped and probed with labeled P actin cDNA probe.

Utilizing virtual Northern analysis, SAC expression was identified in a number of cancers including breast carcinoma (and normal breast), oligodendroglioma, glioblastoma multiforme, astrocytoma, (and normal cerebullum and normal brain), colon adenocarcinoma, prostate carcinoma, and (normal ovary). Accordingly, human SAC may serve as a diagnostic for imaging tumors. In addition human SAC may serve as the basis for the generation of anti-cancer therapies, including the use of antibodies directed against human SAC.

Hydropathy analysis of the encoded protein showed that SAC may be a transmembrane protein containing 5 transmembrane domains with N-terminal intracellular and C-terminal extracellular domains. Interestingly, the adenylate cyclase domains are largely in the N-terminal intracellular domain. This membrane topology remains to be experimentally established.

### EXAMPLE 2

Generation of Antibodies Against the Catalytic Domain of SAC.

The cDNA sequence encoding the SAC catalytic domains (bp 306-1529) was cloned into the pET28b vector and used to express recombinant SAC protein (recSAC) containing a C-terminal His-tag. In particular, primers were designed to create an NdeI site at the 5′ end and an XhoI site at the 3′ end of the catalytic domain. The amplified products were ligated into the NdeI-XhoI sites of pET28b, a 6 His-tag expression vector. Recombinant SAC (recSAC) was purified by immobilized metal (Ni) affinity chromatography and used to generate rat antibodies for Western blot analysis and immunolocalization of the human SAC protein in testis and sperm.

Two separate approaches were used to generate antibodies against human SAC. In the first approach recombinant human SAC purified by chromatography was used to immunize rats. In a second approach a stretch of 16 amino acids (CTLKPDPELEMSLQKY; SEQ ID NO: 5) from the catalytic domain of SAC were uscd to generate antipeptide antibodies in rabbits. These antisera were custom prepared by Biosource International/QCB Division, MA. Both polyclonal antibodies, the rat generated anti rSAC and the rabbit anti peptide antibody recognized recombinant catalytic SAC in western blots. Both antibodies also recognized a 100 kDa band in human sperm extracts that is not observed when the respective preimmune sera were used.

To validate the specificity of these antibodies, immunoreactive proteins will be cut from two dimensional gels and microsequenced. Once validated, the specific antibodies against human SAC will be used in immunofluorescence experiments and immunoelectromicroscopy to investigate the subcellular localization of this enzyme. Since sperm are compartmentalized cells, the localization of human SAC will give information about whether this enzyme has a role in motility, in the events that precede the acrosome reaction or in the regulation of both events.

In order to perform immunolocalization and immunoblotting experiments human sperm will be collected from healthy donors and purified using Percoll (Pharnacia Biotech, Upsala, Sweden) density gradient centrifugation as previously described (Naaby-Hansen, Flickinger et al. 1997). Sperm will be then resuspended to a final concentration of 2×10⁷ cells/ml. In particular for SDS-PAGE and immunoblotting sperm will be pelleted by centrifugation, washed in 1 ml of phosphate buffered saline (PBS), resuspended in sample buffer (Laemmli 1970) without mercaptoethanol and boiled for 5 min. After centrifuging, the supernatant will be saved, 2-mercaptoethanol will be added to a final concentration of 5%, boiled for 5 min., and then subjected to 10% SDS-PAGE. Protein concentration will be determined by ABC kit from Pierce. For tissue specificity at protein level commercially available multiple tissue protein blots will be used. Electrophoretic transfer of proteins to Immobilon P and immunodetection will be carried out as previously described (Kalab, Visconti et al. 1994, J Biol Chem 269(5): 3810-7). Gels will be stained either with silver, coomasie blue or will be transferred to immobilon PVDF (Millipore) and probed with anti recombinant soluble testicular adenylyl cyclase antibodies.

For isolating sperm proteins for two dimensional gel analysis, human sperm will be solubilized in a lysis buffer and isoelectrofocusing will be performed followed by two-dimensional SDS-PAGE using linear gradients as described previously (Naaby-Hansen, Flickinger et al. 1997, Biol Reprod 56(3): 771-87). Gels will be transferred to Immobilon P and blot with anti SAC antibodies. Protein(s) recognized will be cut and microsequenced using the University of Virginia W. M. Keck Biomedical Mass Spectrometry laboratory facility. In order to identify the exact location of an immunoreactive antigen in the complex 2Dprotein pattern, antibody incubations will be preceded by staining with Protogold (Goldmark Biologicals, Phillipsburg, N.J.) following the manufacturer's instructions.

For immunofluorescence analysis sperm will be treated in the appropriate experimental conditions, fixed in suspension with a solution of 3% (w/v) paraformaldehyde-0.05% (v/v) glutaraldehyde in PBS for 1 h, washed in PBS at 37 C, and then permeabilized with 0.1% (v/v) Triton X-100 in PBS at 37 C for 10 min. The sperm will be then washed in PBS and incubated overnight with serial dilutions (5, 10, 50 and 100) of the appropriate antibody. After washing the sperm with PBS, they will be incubated with FITC-coupled goat anti mouse IgG and then attached to poly-lysine-coated microscope slides. Following 3 X washes with PBS, the slides will be mounted with fluoromont and fluorescence will be assessed.

Experiments from Chen and coworkers demonstrated that the enzymatic activity of rat SAC could be modulated by bicarbonate. It is expected that human SAC will be also regulated by this anion. To demonstrate this hypothesis the full length and the catalytic domain will be expressed in bacteria and/or yeast and cyclase activity will be measured. The activity will be measured in the presence or absence of different concentrations of bicarbonate (1, 5, 10, 15, 20, 30 and 50 mM) as described previously (Visconti, Moore et al. 1995 Development 121(4): 1139-50).

### EXAMPLE 3

Isolation of Specific Inhibitors of the Human Soluble Testicular Adenylyl Cyclase.

Adenylyl cyclase is a central component of signaling pathways both in the sperm and in the testis, and therefore specific inhibitors of this enzymatic activity are likely to inhibit sperm's ability to fertilize and/or the spermatogenic process. Since the soluble testicular cyclase has a unique sequence that differs from somatic adenylyl cyclase it should be possible to find specific inhibitors of this soluble cyclase that do not affect other enzymatic activities.

To identify enzyme inhibitors of human SAC two different approaches will be utilized. In the first approach an assay is used that allow the screening of thousand of compounds for compounds that inhibit human SAC activity. In addition to this approach the crystal structure of the enzyme will be determined and inhibitory compounds will prepared based on that crystaline structure. For both approaches it is necessary that sufficient quantities of the enzyme be available and that the enzyme be correctly folded.

The catalytic domain of the human soluble testicular adenylyl cyclase has been expressed in bacteria and this protein was used to generate antibodies in rats. An independent validation of the expressed protein was performed using the antipeptide antibody against SEQ ID NO: 5 (described in Example 1). Recombinant catalytic SAC was found to be present in the bacterial insoluble fraction. Before attempting to produce high amounts of the recombinant domain of SAC, the enzyme will be solubilized in urea and refolded by dialysis against an isotonic buffer. This buffer consists on 150 mM NaCl, Tris/HCl 50 mM pH 7.5, protease inhibitors (leupeptin 10 ug/ml and aprotinin 10 ug/ml) and a small concentration of detergent (triton X100 0.1%). The urea concentration will be reduced in several steps to maximize the correct refolding of the protein. The presence of Triton has already demonstrated that it does not affect sperm cyclase enzymatic activity. The correct refolding of recombinant SAC catalytic domain will be tested using a cyclase assay. If the refolded enzyme is active it will also be tested for the regulation with bicarbonate anion.

A similar approach will be taken to express human SAC in yeasts. In summary, the catalytic domain will be subcloned in pPICZaB vector from Invitrogen (CA) and will be expressed in Pichia pastoris as secreted protein as well as an intracellular protein. These constructs have a C-terminal His tag that will allow an easy purification of the recombinant protein either from the culture media or from the extracted cells.

Measuring Adenylyl Cyclase Activity in Vitro

In one embodiment, adenylyl cyclase activity will be measured by the conversion of [α−³²P] ATP to [³²P] cAMP. The assay will be carried out for 20 min. at 37° C. in the presence of 50 mM Hepes, 1.5 mM MgCl₂ or MnCl₂, 10 mM KCl, 4 mM DTT, 1 mM 3 MX, 16 μg creatine kinase, 3.2 mM creatine phosphate and 2.5 μCi (α−³²P} ATP, pH 7.6 in a final volume of 30 ml. The assay will be started by addition of 8 μl of cell suspension containing 0.5-1.2 μg of protein, and will be terminated by the addition of 25 μl of the termination buffer (36.4 mM ATP, 10 mM cAMP, 1% SDS and 300 cpm [³H] cAMP) followed by heating in boiling water batch for 5 min. The [³²P] cAMP will be purified following chromatography using Dowex and Alumina columns and the [³H] cAMP will be used as an internal standard for the evaluation of recovery.

The preferred method, especially for high throughput screening, for measuring the cAMP synthesized by recombinant SAC will be through the use of an radioimmunoassay (RIA). Briefly, fluorescently labeled anti cAMP antibody will be excited by iodinated cAMP only when cAMP is bound to the antibody. By competing the iodinated cAMP with the cAMP synthesized in the assay, it is possible to measure cyclase activity in a single tube. This methodology has been previously described in the literature at Steiner, et al., 1972 J. Biol. Chem. 247: 1114-1120 as modified in Visconti and Tezon, Biol. Reprod., 1989 40: 223-231. lodinated cAMP is commercially available through New England Nuclear and from Amersham

Determining the Crystal Structure of the Catalytic Domain of the Human SAC

Approximately five milligrams of protein is generally deemed a suitable quantity for initial crystallization trials. It is envisaged that initial trials will be performed on the catalytic domain of the intact protein; this is a common crystallization technique that has proven to be successful in many cases, including several structural studies of soluble mammalian cyclases. Prior to crystallization, the expressed fragment would be screened for a suitable level of enzymatic activity as well as for purity, homogeneity and high solubility. Several commercially available crystallization screening kits, used in conjunction with the hanging drop vapor diffusion method, provide the standard first step in the search for crystal growth conditions. Crystallization screening of the fragment in the presence of catalytically required metal ions, substrates and/or inhibitors will be carried out simultaneously with the screen of the apoenzyme. In the case of the soluble adenylyl cyclase, crystallization in the presence of bicarbonate will be attempted in order to establish a molecular basis for the stimulatory properties of this anion.

If necessary, other standard screening methods may be employed. If these do not result in promising conditions, a reevaluation of the biophysical solution properties of the fragment may point to a modification of the construct used for expression. Once initial crystallization conditions are obtained, they will be optimized in order to obtain crystals that diffract to at least 3.0 A resolution. Direct phasing of the structure via the MAD (multiple wavelength anomalous dispersion) and MIR (multiple isomorphous replacement) techniques would be carried out simultaneously, to guarantee success. Phasing via the molecular replacement technique would likely fail due to the lack of sufficient sequence identity between the testis-specific cyclase and the structurally characterized cyclases. Semi-automated model-building and refinement techniques for phased structures would be utilized to achieve rapid structural results. Once a structure is obtained the results will be analyzed with a view to understanding the unique role of the soluble testicular cyclase in sperm capacitation. The crystalline structure will be used as a template for the design of specific inhibitors of testicular cyclase, which may prove useful as contraceptives.

1. An isolated polypeptide comprising an amino acid sequence selected from the group consisting of SEQ ID NO: 3 or SEQ ID NO:4.

2. The polypeptide of claim 1 wherein the amino acid sequence is SEQ ID NO: 3 or SEQ ID NO: 4.

3. A purified polypeptide having adenylyl cyclase activity and comprising an amino acid sequence that differs from SEQ ID NO: 3 by one or more conservative amino acid substitutions.

4. A nucleic acid sequence comprising the sequence of SEQ ID NO: 2.

5. A nucleic acid sequence that hybridizes to a 100 nucleotide fragment of SEQ ID NO: 2 under stringent conditions.

6. A transgenic host cell comprising the nucleotide sequence of claim 4.

7. An isolated antibody that binds specifically to the polypeptide of SEQ ID NO: 3.

8. The antibody of claim 8 wherein the antibody is a monoclonal antibody.

9. A composition comprising a human adenylyl cyclase and a pharmaceutically acceptable carrier.

10. A method for detecting compounds that inhibit human soluble adenylyl cyclase activity, said method comprising the steps of
contacting a polypeptide, comprising an amino acid sequence selected from the group consisting of SEQ ID NO: 3 and SEQ ID NO: 4, with a compound;
measuring adenylyl cyclase activity in the presence of said compound; and identifying compounds that decrease the activity of adenylyl cyclase.

11. The method of claim 10 further comprising the step of testing said identified compounds for inhibitory activity against a somatic adenylyl cyclase.

12. The method of claim 11 wherein adenylyl cyclase activity is determined by radioimmunoassay measurements of cAMP formed from ATP.
